(19) United States
(12) Patent Application Publication
(10) Pub. No.: US 2023/0381550 A1
(43) Pub. Date: Nov. 30, 2023

(54) FACE MASK WITH BATTERY OPERATED FAN AND UV BULB

(71) Applicant: Gary Austin Firlotte, Edmonton (CA)

(72) Inventor: Gary Austin Firlotte, Edmonton (CA)

(21) Appl. No.: 17/828,427

(22) Filed: May 31, 2022

(51) Int. Cl. A62B 18/02; A62B 18/08; A62B 18/00; A61L 9/20; A62B 23/02

(52) U.S. Cl. CPC A62B 18/025; A62B 18/084; A62B 18/006; A62B 18/086; A61L 9/20; A62B 23/02

(57) ABSTRACT

Embodiments of the invention relate to a solar or battery operated cooling fan assembly attached to face mask. The fan allows venting of warm moist breath vapors, reducing heat, heat rash, reduces low energy due to CO2, circulates air, thereby cooling the user. The fan assembly also may comprise a UV light bulb, that sterilizes exhaled air as it exits the mask. The face mask includes an inner framework or structure to support the fan assembly. The face mask may also include one or more filters to allow air to enter the mask, and in some embodiments straw exit and entry ports are provided.

### FIELD OF THE INVENTION

The present invention relates generally to the field of personal protective equipment, specifically to single or multiple use face masks, more specifically to face masks having a solar or battery-operated fan and UV air sterilization capabilities.

### BACKGROUND

Canadian Patent 2,374,925 (Bowen) discloses a face mask with fan attachment. A face mask is provided having a filter body sized to cover the nose and mouth of a wearer, a securing mechanism for holding the filter body in close fit with the wearer's face, and a fan attachment to enhance breathability and comfort of the mask. The fan operates to draw warm, moist exhaled air from the interior volume of the mask to improve comfort, facilitate inhalation, and decrease fogging of any transparent portions of the mask or eye coverings. The fan may additionally operate to force cool, fresh air into the interior volume of the mask, also for the comfort of the wearer. The fresh air that enters the mask, however, is not sterilized prior to entry, so the mask does not protect the wearer from infection.

U.S. Pat. App. 2015/0202473 (Curran) discloses a powered exhaust apparatus for a personal protection respiratory device. The device relates to an exhaust apparatus for releasable or permanent connection to a personal protection respiratory device that defines a filtered air volume adjacent to the face of a wearer and comprises at least one exhalation valve. The exhaust apparatus comprising a powered blower in fluid connection with the at least one exhalation valve, the blower being operable to draw a portion of the wearer's exhaled breath through the at least one exhalation valve. Using such an exhaust apparatus for releasable connection to a personal protection respiratory device improves the comfort and overall experience for respirator wearers who use the respirator for intensive work, and/or for long periods of time, and/or in hot and humid environmental conditions by removing the heat and moisture build-up inside the respirator.

U. S. Pat. App. 2003/0066527 (Chen) discloses a face mask having a device for drawing air into the mask. A face mask includes a fan device for drawing air into the mask and for facilitating the breathing of the users. A holder is secured to the mask and has a chamber for receiving the fan device. A motor may be used for driving the fan device. A canister is disposed in front of the fan device for filtering air. A protective device or a guard member is further provided and disposed between the canister and the fan device, and includes a passageway formed and provided therein for allowing the air to be drawn through the canister by the fan device, and for allowing the air to flow into the holder.

U.S. Pat. No. 5,372,130 (Stern) discloses a face mask assembly and method having a fan and replaceable filter. The face mask assembly comprises a main housing and a replaceable filter removably mounted to the housing so that with the assembly being worn by the person, the filter is positioned to define with the person's facial breathing area an air breathing region from which the person inhales air and into which the person exhales air. The filter is arranged to have outside air pass therethrough into said air breathing region to be inhaled. A fan means draws air into a plenum chamber defined by said housing and said filter for this air to pass into a facial breathing area. An outlet check valve is provided, and a primary filter through which air passes before entering said plenum chamber.

U. S. Pat. App. 2019/0275359 (Shen) discloses a custom-controllable powered respirator face mask. The embodiments of the invention relate to respirator face masks, and specifically to powered face masks which may be custom-controllable to better provide for the specific air needs of the individual user wearing the mask. For example, the face mask embodiments typically include a filter and a motorized fan, both generally located on the face mask itself, along with a processor.

EP 3,574,959 (Weizhong) discloses a breathing assistance face mask and method of its control. The invention relates to breathing assistance face masks in which an electric fan draws air through an air filter into the mask or draws air out of the mask, to assist inhalation or exhalation. There may be two fans, operable to assist respectively in the exhalation and inhalation of air through the filter or through respective filters. They may be timed by the same controller. A power source such as a battery, preferably rechargeable, is arranged in the mask to power the control unit, pressure sensor arrangement and the fan.

KR 102117442 (Sun Hee) discloses a mask having a fan. The mask provided with the fan includes an inner frame; an outer frame detachably coupled to the outside of the inner frame; a first and second soft frames made of a soft material that is coupled to the upper and lower parts of the inner frame and is fixed by the outer frame and is in contact with the skin, and a mask filter provided between the inner frame and the outer frame. Wherein the inner frame is provided with an exhaust fan for discharging inner air to the outside. According to the present invention, by providing an exhaust fan portion on the inner frame, carbon dioxide contained in the exhalation can be quickly and smoothly discharged to relieve discomfort of breathing.

CN 106102839 (Mee Young) discloses a mask. The mask, includes a body and an air purifier. The air purifier is connected to the body or is formed on body and be configured to purify introduced air therein with discharge. The control unit is connected on the body or is formed on body, and is configured so that the air is purified and discharged by air purifier. The air purifier includes an air intake and filter unit, it includes being configured for purification passing through at least one filter for the air intake is introduced. The blower fan, it is configured to adjust when rotated so that the air through filter unit that is introduced by air intake, discharged by air outlet slit. Control unit is based on the vibration of mask or the air capacity of extraneous air control the rotary speed of blower fan.

U. S. Pat. App. 2003/0154983 (Marx) discloses a personal air filtering device. The device has a face mask comprised of nonporous material. The face mask held onto the user's head by standard elastic straps. The face mask having a flexible gasket about its perimeter to provide an airtight seal between the mask and the user's face. The face mask having a check valve that lets exhaled air out of the mask but does not let unfiltered air into the mask, a flexible tube that enters the face mask at one end and enters an air filtering assembly on the opposite end, said air filtering assembly enclosed in a housing, said housing having an air intake opening, and said air filtering assembly including an air moving device such as a fan or pump, a carbon filter cartridge, a particulate matter filter, a battery power supply, and an on-off switch. A preferred embodiment includes said flexible tube held onto the user's clothing by a neck clip.

All documents cited herein are incorporated by reference.

None of the above cited documents, alone or in combination satisfy the need for to single or multiple use face masks, having solar or battery-operated fan and UV air sterilization capabilities.

### BRIEF SUMMARY

It is an object of the invention to provide a face mask and battery-operated fan and UV bulb.

In accordance with an aspect of the invention there is provided a face mask comprising: an inner cover, sized and configured to extend from ear to ear and chin to bridge of nose of a wearer, said inner cover having an inner surface and an outer surface; an outer cover connected to said inner cover, said outer cover being sized and configured to extend from ear to ear and chin to bridge of nose of said wearer, said outer cover having an inner surface and an outer surface and a void positioned in close approximation to mouth of said wearer; a support structure, positioned between said outer surface of said inner cover and said inner surface of said outer cover, said support structure comprising one or more support members; a fan assembly comprising a fan and power source, said assembly configured extend through said void and attach to one or more of said one or more support members; and two ear straps attached to said inner cover or said outer cover of said face mask, wherein said two ear straps loop around the ears of said wearer and hold the face mask in position from ear to ear and chin to bridge of nose of said wearer.

In accordance with another aspect of the invention there is provided a face mask as described above wherein said fan assembly and said one or more of said one or more support members are configured to contain cooperating releasable fittings selected from the group comprising: push-fit connections; bayoneted connections; screw threaded connections; and friction fit connections.

In accordance with an additional aspect of the invention there is provided a face mask as described above wherein said fan assembly additionally comprises a UV bulb that is configured to be powered by said power source to emit short wavelength ultraviolet light.

In accordance with yet an additional aspect of the invention there is provided a face mask as described above wherein one or more of said one or more support members comprises a straw port and said inner cover and said outer cover each have resealable voids in the approximate vicinity of said straw port.

The advantages and features of the present invention will become better understood with reference to the following more detailed description and claims taken in conjunction with the accompanying drawings in which like elements are identified with like symbols.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a front aspect of the support structure of the face mask in accordance with one embodiment of the invention.

FIG. 2 illustrates a perspective side aspect of the support structure of the face mask in accordance with one embodiment of the invention.

FIG. 3 illustrates a front aspect of the support structure of the face mask in accordance with one embodiment of the invention.

FIG. 4 illustrates a perspective side aspect of the inner cover in accordance with one embodiment of the invention.

FIG. 5 illustrates a perspective side aspect of the front section of the face mask in accordance with one embodiment of the invention.

FIG. 6 illustrates a perspective side aspect of the front section of the face mask and fan assembly in accordance with one embodiment of the invention.

FIG. 7 illustrates perspective front view aspects of the fan assemblies and battery casing in accordance with one embodiment of the invention.

FIG. 8 illustrates a top aspect of the nose protector in accordance with one embodiment of the invention.

FIG. 9 illustrates a front aspect of the straw entry port in accordance with one embodiment of the invention.

FIG. 10 illustrates a front of the straw exit port in accordance with one embodiment of the invention.

FIG. 11 illustrates a front of the straw exit port in combination with an external flap in accordance with one embodiment of the invention.

### DETAILED DESCRIPTION

Devices and methods for carrying out the invention are presented in terms of embodiments depicted within the FIGS. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

Embodiments of the invention relate to a battery-operated cooling fan assembly attached to face mask. The fan allows venting of warm moist breath vapors, reducing heat, heat rash, reduces low energy due to CO2, circulates air, thereby cooling the user. The fan assembly also may comprise a UV light bulb, that sterilizes exhaled air as it exits the mask. The face mask includes an inner framework or structure to support the fan assembly and power source. The face mask may also include one or more filters to allow air to enter the mask, and in some embodiments straw exit and entry ports are provided.

The features of the invention which are believed to be novel are particularly pointed out in the specification. The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

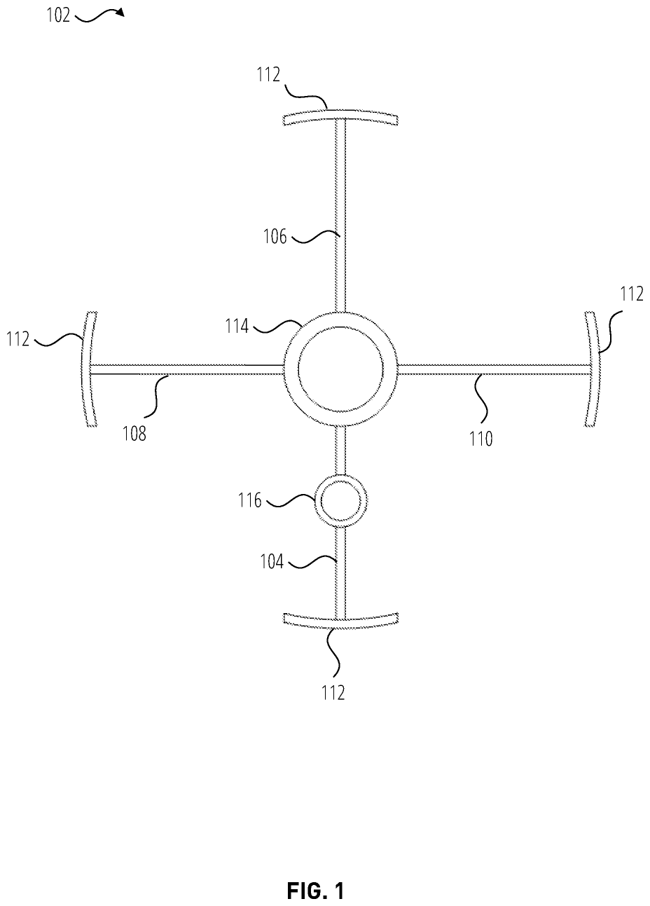

FIG. 1 shows the inner supporting framework that supports various features of the face mask.

The support structure 102 can be seen in this embodiment of the invention to comprise a lower vertical support 104, an upper vertical support 106, a first side support 108, and a second side support 110. One or more support mounts 112 can be seen attached to the outer end of the lower vertical support 104, the upper vertical support 106, the first side support 108, and the second side support 110. The function of these support mount 112 features is to assist in securing the support structure 102 to the face mask.

The support structure 102 can consist of a plurality of structural members, typically fabricated from plastic. The structural members function to: extend the front of the face mask away from the face of the wearer; and to support the fan assembly and battery casing. In one embodiment of the invention, the structural members can extend from the fan assembly to the edges of the mask, in other embodiments, the structural members can extend a lesser distance (as illustrated in FIG. 1).

The support structure 102 provides a structure shown here as a fan mount 114 that is positioned on either the upper vertical support 106 of the lower vertical support 104. The embodiment of the invention illustrated here also includes a feature shown here as a straw port 116 that enables the wearer to insert a straw through the straw port 116.

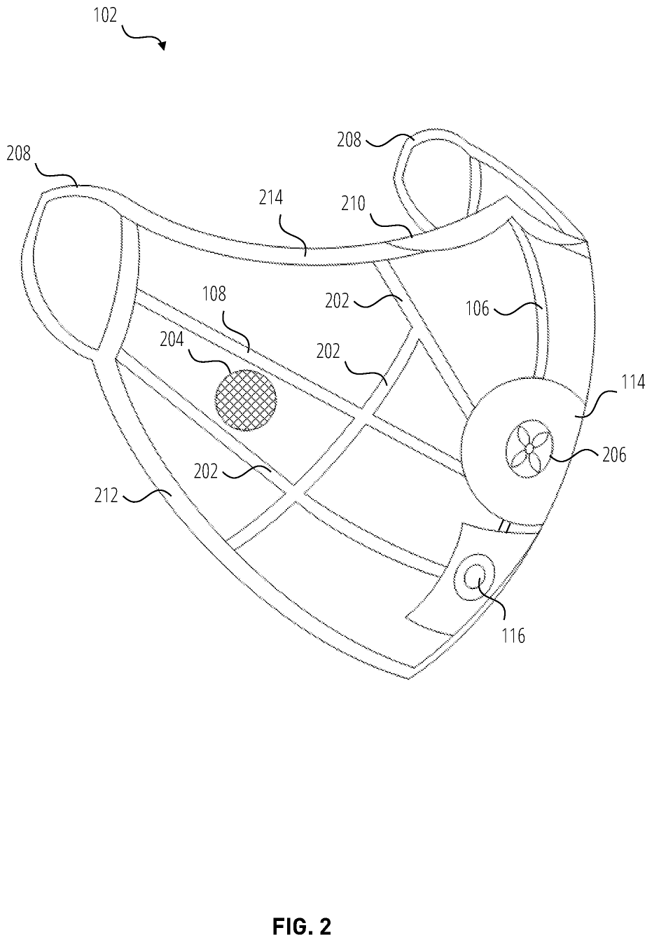

FIG. 2 shows an alternative embodiment of the inner supporting framework that supports various features of the face mask.

In this illustrated embodiment, the support structures 102 of the mask can be seen extending to the edges of the mask. The upper vertical support 106, a first side support 108, and straw port 116 can also be seen. A plurality of structural members, indicated generally as additional supports 202 are also shown.

The positioning of a filter 204 can be seen on the side of the support structure 102 which allows air to enter into the face mask. The relative positioning of a fan 206 can be seen at the front of the mask. The mask further comprises a lower mask edge 212 and an upper mask edge 214. Ear straps 208 can be seen positioned at either edge of the mask, and in some embodiments of the invention, a nose protector 210 may be present on the upper mask edge 214 in the region of the nose.

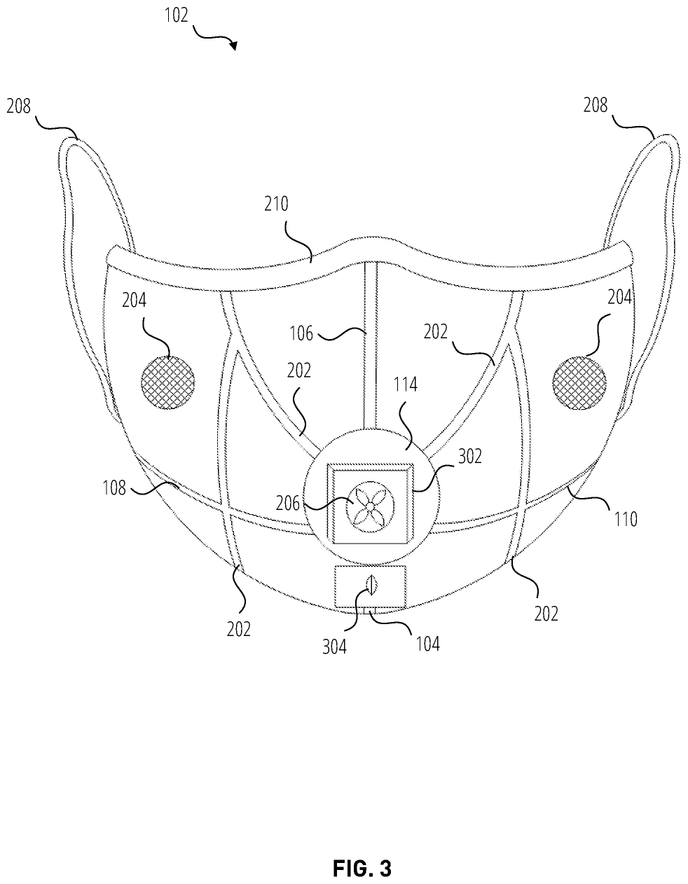

FIG. 3 shows a front view of the support structure 102 of the face mask.

The lower vertical support 104, upper vertical support 106, first side support 108 and second side support 110 can be seen, along with additional supports 202. The fan mount 114 is seen supporting a fan assembly 302 comprising a fan 206 and necessary power source and mechanical features (not shown).

Filters 204 can be seen positioned essentially on the cheek area of the face mask embodiment illustrated herein. The ear straps 208, and nose protector are positioned as shown in FIG. 2. An external straw flap 304 can be seen overlaying the straw port 116 (not shown).

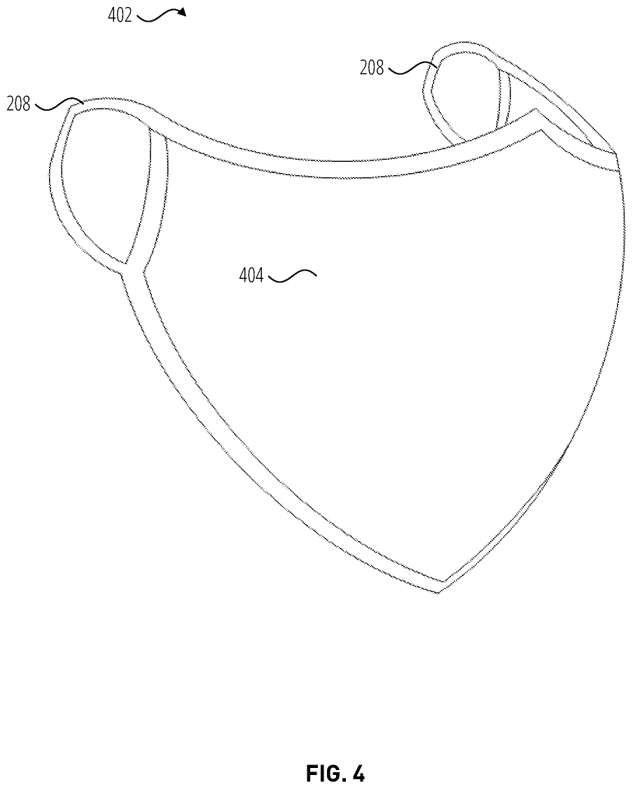

FIG. 4 shows a face mask 402 in accordance with an embodiment of the invention.

The face mask 402 shows an inner cover 404 and ear straps 208.

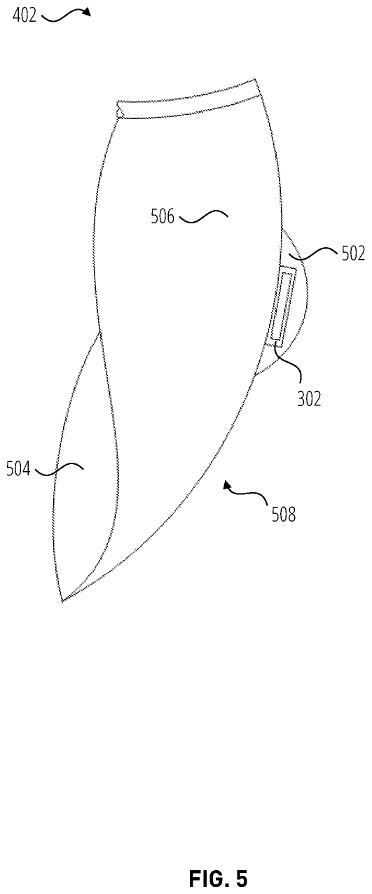

FIG. 5 shows a front section of the face mask 402 having an inner surface 504 and an outer surface 506. The fan assembly 302 can be seen incorporated within a fan casing 502.

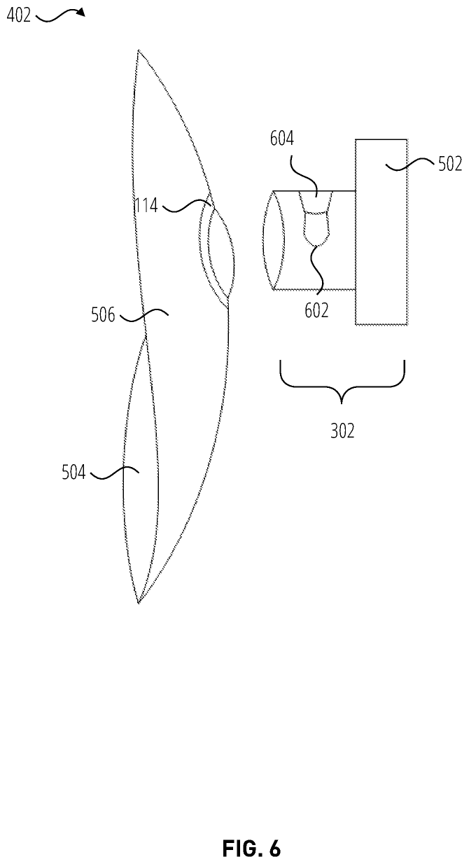

FIG. 6 shows a face mask 402 having a removable fan assembly 302 for attachment or fitting to a fan mount 114. The removable fan assembly 302 can be attached to the fan mount 114 (not shown) by a variety of fittings known to those of skill in the field including, but not limited to: push-fit connections; bayoneted connections; screw threaded connections; and friction fit connections.

The face mask 402 illustrates an inner surface 504 and outer surface 506. In this particular embodiment of the invention, the fan assembly 302 comprises a UV bulb 602 affixed to a UV bulb mount 604. The fan 206 is present within the fan casing 502. In other embodiments of the invention, the UV bulb 602 may be positioned on the opposite side of the fan 206.

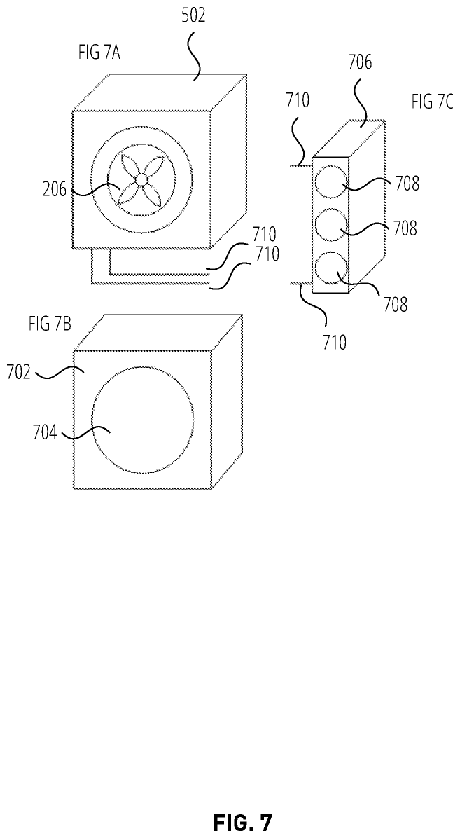

FIGS. 7A, 7B, and 7C show various different embodiments of the fan casing 502 and attachments.

FIG. 7A illustrates a fan 206 positioned within a fan casing 502. Wirings 710 can be seen entering and exiting the fan casing 502 thereby providing a power source to propel the fan.

FIG. 7B illustrates an embodiment of the UV casing 702 (UV bulb 602 not shown). The air that leaves exits the interior of the mask exits the mask via the exhaled air exit 704.

FIG. 7C illustrates the battery case 706 that is sized and proportioned to fit onto an exterior surface of the face mask 402. In the embodiment of the invention shown, the battery case 706 attaches to an exterior surface of the fan casing 502. One or more batteries 708 can be seen fitted within the battery case 706. The wirings 710 deliver a DC power supply to the fan 206 and UV bulb 602, thereby driving sterilized exhaled air out of the face mask 402.

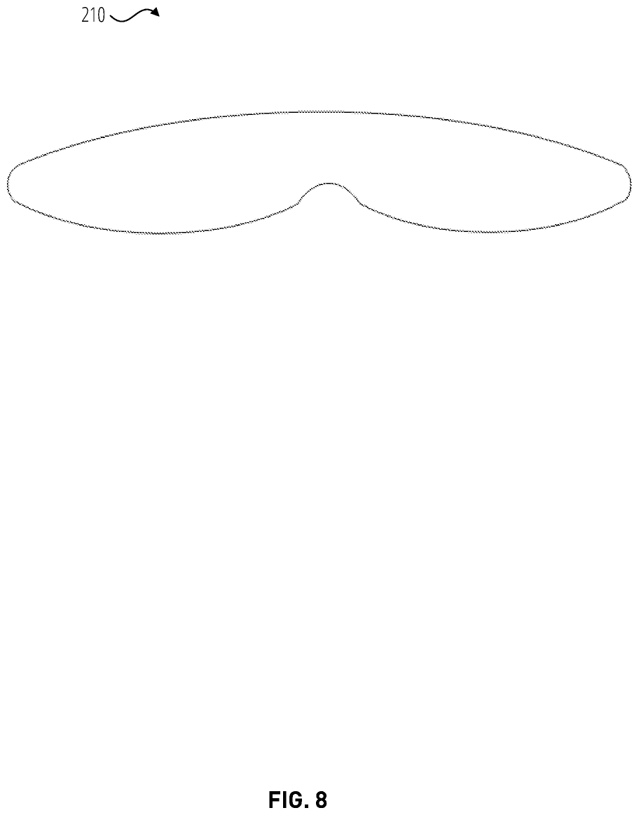

FIG. 8 shows an embodiment of a nose protector 210 that can be attached to the upper mask edge 214. The nose protector 210 is typically made from a soft and flexible material such as silicone or a fabric. For reusable embodiments of the invention, the nose protector 210 is made from washable materials.

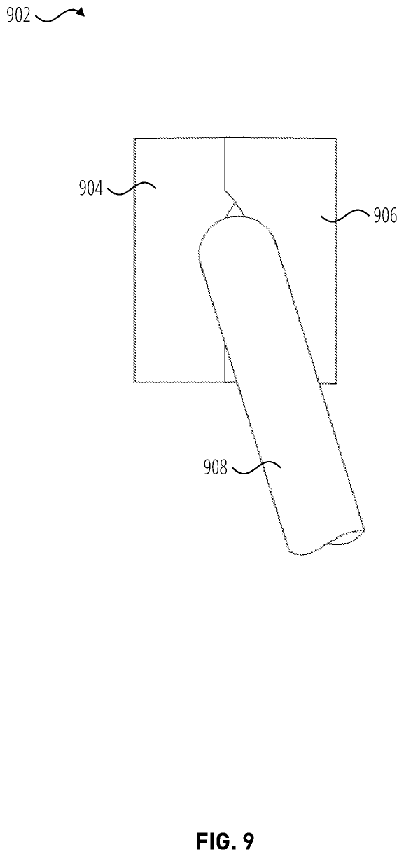

FIG. 9 shows the exterior surface of the area of the mask surrounding the straw entry port 902. In the embodiment illustrated herein, a first fabric overlap 904 and a second fabric overlap 906 create a flexible overlapping cover allowing straw 908 to be inserted between the first fabric overlap 904 and second fabric overlap 906 and through the straw port 116 (not shown) such that the wearer can easily take a drink without removing the face mask 402.

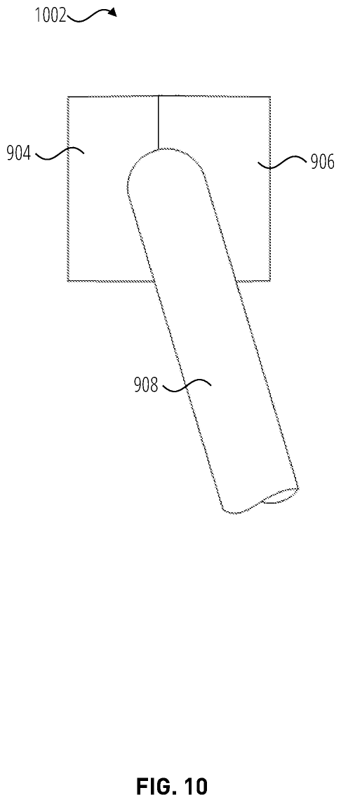

FIG. 10 shows the exterior surface of the area of the mask surrounding the straw exit port 1002. In the embodiment illustrated herein, the first fabric overlap 904 and the second fabric overlap 906 create a flexible overlapping cover for the straw port 116 (not shown) when not in use. When the straw 908 is removed, after the wearer has taken a drink, the first fabric overlap 904 and second fabric overlap 906 reseal to cover the straw port 116 and once again render the face mask 402 sealed from air leakages.

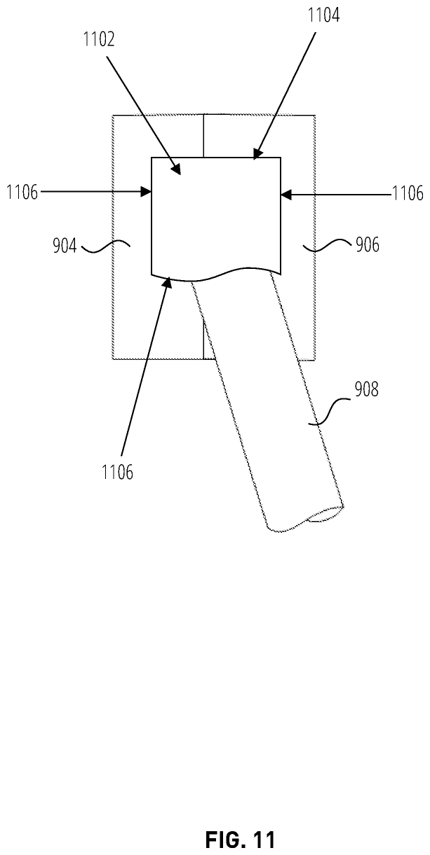

FIG. 11 shows that in some embodiments of the invention, the drinking port comprises two or more pieces of overlapping elastic material 904 and 906, on an inner surface of the mask. On the outer surface of the mask, and over the overlapping pieces of elastic material that from the straw port 116 (not shown), a flap of material can be attached. The flap 1102 may be permanently attached 1104, such as by sewing along one edge and may be releasably attached 1106, along the other edges using, for example hook and loop fasteners. Releasing the releasably attached 1106 perimeter of the flap thereby allowing for insertion of a straw 908.

As described above in conjunction with the figures, embodiments of the face mask 402 may contain various different features.

The cooling fan assembly 302 that is attached to the face mask 402 can be powered by a variety of different batteries 708. It is important to reduce the weight of the attachments to the face mask 402, hence AAA lithium or alkaline batteries are suitable source of power. However, silver oxide batteries, commonly used in wrist watches and other small devices, are considered a suitable alternative power source.

Similarly, the UV bulb 602 that is incorporated into the fan casing 502 in some embodiments of the invention is powered by the power supply that powers the fan 206. In the currently described system, the UV irradiation used is short wavelength ultraviolet (ultraviolet C or UV-C) light. Hydroxyl radicals are produced in the exhaled moist air by ultraviolet irradiation. The hydroxyl radicals that are produced by the ultraviolet C or UV-C light then kill or inactivate organic material and microorganisms such as bacteria or viruses. This is achieved by destroying nucleic acids and disrupting the DNA of the microorganism, leaving them unable to perform vital cellular functions thereby rendering the exhaled air sterile prior to exiting the face mask 402.

The face mask 402 includes an inner framework or support structure 102 to support the fan assembly 302 and power source such as a battery case 706. The support structure 102 can consist of a plurality of structural members, typically fabricated from plastic. The structural members function to: extend the front of the face mask 402 away from the face of the wearer; and to support the fan assembly 302, UV bulb mount 604, UV bulb 602 and battery UV casing 702. In one embodiment of the invention, the structural members can extend from the fan assembly to the edges of the mask, in other embodiments, the structural members can extend a lesser distance. The support structure 102, in particular the support mounts 112, also assist in anchoring the attachments to the face mask 402 to the inner cover 404 and/or outer cover 508 in the appropriate position close to the mouth on the face mask 402. In some embodiments of the invention, a small solar panel can be situated on the upper surface of the fan casing 502. The solar energy can then power the fan 206 and the UV bulb 602. It is contemplated that this embodiment may be suitable for persons working in close contact with other people in outdoor settings, such as catering at outside functions, or ushering crowds at outdoor events, etc. The solar power can directly power the fan and UV light, or it can be stored in a battery.

The face mask 402 includes an inner cover 404 that is essentially a continuous piece of fabric. The inner cover 404 is typically porous to air flow and as such can cover the inner surface of the fan mount 114 in certain embodiments of the invention. The function of the inner cover 404 is to: prevent the support structures 102 from chafing the face; to provide a possible anchor point for the support structures 102; and to prevent the UV light entering into the mask and causing a distraction to the wearer. In some embodiments of the invention, a void or opening can be removed from the inner cover 404 in the vicinity of the fan mount 114 in order to increase air flow through the exhaled air exit 704 in the embodiments that also have the UV bulb 602 to sterilized the exhaled air.

The face mask 402 also includes an outer cover 508 that is essentially a continuous piece of fabric that is typically impervious to air flow. The outer cover can be made from a flexible or expandable material such as Spandex™ or a waterproof material. The inner cover 404 has a void or opening for the fan assembly 302, and when present also the straw entry port 902 and one or more filters 204. The function of the inner cover 404 is to provide a possible anchor point for the support structures 102; and to allow customization of color or design. In the embodiments of the invention, having the straw entry port 902, the outer cover 508 either forms the first fabric overlap 904 and second fabric overlap 906 or it provides an attachment or fitting point for the first fabric overlap 904 and the second fabric overlap 906.

The face mask 402 may also include one or more filters 204 to allow air to enter the face mask 402. The filter 204 or filters 204 may be attached to the support structure 102 and a void or opening removed from the outer cover 508. Or, in alternative embodiment of the invention, the filter 204 or filters 204 may be attached to an outer surface of the outer cover 508, overlying the void or opening that has been removed from the outer cover 508.

Typically, some form of binding is applied to the upper mask edge 214 and lower mask edge 212 to connect the outer cover 508 with the inner cover 404. The binding may also extend to form ear straps 208, or some other form of material, such as elastic, may be applied to the outer edges of the face mask 402 to form ear straps 208.

In some embodiments of the invention, a nose protector 210 may be permanently or releasably attached to the upper mask edge 214. The nose protector 210 is typically made from a soft and flexible material such as silicone or a fabric.

It is contemplated that the face mask 402 as described herein can be fabricated for single use. The fan assembly 302 being releasably attached to the fan mount 114 using various fittings as described above. Such embodiments of the invention would likely be made from paper or readily degradable materials. Preferred materials would be recyclable, biodegradable, compostable and therefore easily disposable in an environmentally friendly manner. Such materials can include, but are not limited to, paper, compacted paper or card, polypropylene, bamboo, bioplastics, soy bean, potato, avocado, nopal and other vegetable, plant or fruit-based plastics, mycelium polymer-based materials, seaweed polymers and hemp.

It is also contemplated that the face mask 402 can be made to be reusable. Such embodiments of the invention would be made from washable materials. The fan assembly 302 being releasably attached to the fan mount 114 using various fittings as described above.

The foregoing descriptions of specific embodiments of the present invention have been presented for purposes of illustration and description. They are not intended to be exhaustive or to limit the invention and method of use to the precise forms disclosed. Many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

1. A face mask comprising:
an inner cover, sized and configured to extend from ear to ear and chin to bridge of nose of a wearer, said inner cover having an inner surface and an outer surface;
an outer cover connected to said inner cover, said outer cover being sized and configured to extend from ear to ear and chin to bridge of nose of said wearer, said outer cover having an inner surface and an outer surface and a void positioned in close approximation to mouth of said wearer;
a support structure, positioned between said outer surface of said inner cover and said inner surface of said outer cover, said support structure comprising one or more support members;
a fan assembly comprising a fan and power source, said assembly configured extend through said void and attach to one or more of said one or more support members; and
two ear straps attached to said inner cover or said outer cover of said face mask,
wherein said two ear straps loop around the ears of said wearer and hold the face mask in position from ear to ear and chin to bridge of nose of said wearer.

2. The face mask of claim 1, wherein said fan assembly and said one or more of said one or more support members are configured to contain cooperating releasable fittings.

3. The face mask of claim 2, wherein said cooperating releasable fittings are selected from the group comprising: push-fit connections; bayoneted connections; screw threaded connections; and friction fit connections.

4. The face mask of claim 2, wherein said fan assembly additionally comprises a UV bulb that is configured to be powered by said power source to emit short wavelength ultraviolet light.

5. The face mask of claim 4, wherein said UV bulb is mounted in said fan assembly between said mouth of said wearer and said fan.

6. The face mask of claim 1, wherein said power source is provided by a lithium battery, and alkaline battery, a silver oxide battery, or solar power.

7. The face mask of claim 1, wherein said face mask additionally comprises one or more filters.

8. The face mask of claim 7, wherein said one or more filters are mounted on either said outer cover or on one or more of said one or more support members.

9. The face mask of claim 1, wherein one or more of said one or more support members comprises a straw port.

10. The face mask of claim 9, wherein said inner cover and said outer cover each have resealable voids in the approximate vicinity of said straw port.

11. The face mask of claim 10, wherein said resealable voids comprise two overlapping pieces of fabric.

12. The face mask of claim 1, wherein a nose protector is attached to an edge of said face mask in the vicinity of said bridge of the nose.

13. The face mask of claim 12, wherein said nose protector is releasably attached to said edge of said face mask.

14. The face mask of claim 12, wherein a nose protector is made from silicone.

15. The face mask of claim 1, wherein said support structure is fabricated from paper, compacted paper or card, polypropylene, bamboo, bioplastics, soy bean, potato, avocado, nopal and other vegetable, plant or fruit-based plastics, mycelium polymer-based materials, seaweed polymers and hemp.

16. The face mask of claim 1, wherein the outer cover is fabricated from a flexible or expandable material such as Spandex™ or from a waterproof material.

17. The face mask of claim 1, wherein said inner cover, outer cover, support structure and ear straps are fabricated from washable material.

18. The face mask of claim 9, wherein a flap of material is attached an outside surface of the mask positioned over the straw port.

19. The face mask of claim 18, wherein the flap of material is permanently attached to the outer surface of the mask along part of a perimeter of the flap and releasably attached to the outer surface of the mask along part of a remainder of the perimeter of the flap.
